(19) United States
(12) Patent Application Publication
(10) Pub. No.: US 2022/0215951 A1
(43) Pub. Date: Jul. 7, 2022

(54) MEDICAL DEVICE AND METHOD FOR REMOTELY ACCESSING A MEDICAL DEVICE

(71) Applicant: GAMBRO LUNDIA AB, Lund (SE)

(72) Inventors: Camilla JOHANNESSON, Lomma (SE); Mikael LINDHULT, Lund (SE); Niklas MEBIUS, Bunkeflostrand (SE)

(21) Appl. No.: 17/604,803

(22) PCT Filed: Apr. 16, 2020
(86) PCT No.: PCT/EP2020/060645

§ 371 Date: Oct. 19, 2021

(30) Foreign Application Priority Data
Apr. 24, 2019 (EP) 19170765.2

(51) Int. Cl. G16H 40/67; G06F 21/31; G16H 40/40

(52) U.S. Cl. CPC G16H 40/67; G16H 40/40; G06F 21/31

(57) ABSTRACT

The disclosure relates to the field of medical devices, and more specifically to techniques for remotely accessing automated medical devices. According to a first aspect, the disclosure relates to a method for remotely accessing a medical device (10) configured to perform a medical procedure. The method comprises that the remote communication process (PCOMM) performs the steps of configuring (S1) at least one access criterion for remotely accessing the medical device and establishing (S2) a secure connection between the medical device and a remote system. The method further comprises that the remote communication process (PCOMM) performs the steps of receiving (S3) from the remote system, a request to access the one or more medical processes and forwarding (S5a) the request to the one or more medical processes, upon the request fulfilling the configured at least one access criterion for remotely accessing the medical device (10).

### TECHNICAL FIELD

The disclosure relates to the field of medical devices, and more specifically to techniques for remotely accessing automated medical devices in a secure way.

### BACKGROUND

An automated medical device is a machine, whether used alone or in combination, intended by the manufacturer to be used for humans or animals for a medical purpose. Such a medical purpose may include diagnosis, prevention, monitoring, treatment or alleviation of a disease, injury, or handicap.

Automated medical devices are frequently used in the health care sector and are subject to strict regulatory frameworks to ensure that they are safe and efficient.

In general, an automated medical device is operated by a software system (e.g. installed on the medical device), which is a complex system that needs to be carefully designed to mitigate the risk of malfunctions that may have health implications. The software system typically comprises software configured to cause the medical device to perform a medical procedure, such as a medical treatment or medical diagnosis. The software system typically also comprises software configured to perform support procedures associated with the medical procedure, such as maintenance and service functions. In some situations, it may be desirable to control or access a medical device remotely, e.g. to reconfigure the medical device, to perform software updates or to perform tests or for service purposes.

However, providing a remote system with access to a medical device may also involve risks as external parties may try to intercept or otherwise interfere with the communication and take over control of the medical device. Also, a remotely accessible medical device may be subject to overloading attacks, where incoming requests are received at a very high rate, which prevents the critical internal components from performing medical procedures and/or the associated support procedures as intended or specified. Thus, introducing remote access to a medical device may jeopardize safety for patients by making the medical device vulnerable to different types of cyberattacks (i.e. unauthorized access).

In addition, introducing cybersecurity in a medical device is typically challenging, as the performance of the automated medical device while performing a medical procedure and the associated support procedures typically need to be unaffected. Hence, there is a need for improved ways of handling cybersecurity for automated medical devices that are remotely accessible.

### SUMMARY

It is thus an object of the disclosure to avoid limitations of prior art associated with remotely accessing a medical device. In particular, it is an object to provide a system, method, and apparatus to provide cybersecurity in medical devices, while at the same time assuring safety and performance of the medical procedures and associated support procedures that are performed by the medical device.

According to a first aspect, the disclosure relates to a method for remotely accessing a medical device configured to perform a medical procedure, wherein the medical device is controlled by a software system. The software system comprises one or more medical processes involved in the operation of the medical device and a remote communication process that is separate from the one or more medical processes. The remote communication process is configured to manage communication between the medical device and one or more remote systems. Furthermore, the one or more medical processes have higher priority than the remote communication process. The method comprises that the remote communication process performs the steps of configuring at least one access criterion for remotely accessing the medical device and establishing a secure connection between the medical device and a remote system. The method further comprises that the remote communication process performs the steps of receiving from the remote system, a request to access the one or more medical processes and forwarding the request to the one or more medical processes, upon the request fulfilling the configured at least one access criterion for remotely accessing the medical device. In some embodiments the remote communication process and the one or more medical processes are being separated implies that they have separate memory spaces, individual process states and/or are communicating using inter process communication. Thus, by isolating functionality related to communication with a remote system in one (or possibly several) separate processes, security handling of the communication may be simplified and enhanced. As reception and parsing of the remote requests may require substantial processing resources (e.g. processor power and memory), incoming remote requests may potentially starve out the critical internal components from doing their work, e.g. performing the medical procedure or associated support processes. However, by having a remote communication process that operates in its own execution context and has lower priority than other execution contexts, the risk that the critical internal components are starved out is mitigated.

Furthermore, by isolating the functionality, the need to duplicate code is removed. In instances without isolation of functions, the different internal components of the medical device would have to be configured with the appropriate code to process network communication themselves. Furthermore, quality (i.e. level of security) may also be improved as it may be easier to test cybersecurity functionality when it is isolated in one (or a few) process.

In addition, the method provides improved portability as new protocols for external communication can be added in the remote communication process.

The isolation of the remote communication functionality is also beneficial from a software development perspective, e.g. when analysing if cybersecurity requirements are fulfilled. For validation of security requirements, a reviewer may only have to consider the design of a limited part of the software system.

In some embodiments, the method comprises starting up the medical device. During start-up, all requests to access the one or more medical processes are blocked by the remote communication process. Hence, it is not possible to attack the medical device during start-up.

In some embodiments, the method comprises blocking requests to access the one or more medical processes that fail to fulfil the configured at least one access criterion. Hence, unwanted (or unknown/unrecognised) requests may be blocked, thereby mitigating cyber-attacks. Hence, by only accepting the wanted and enabled requests system load may be reduced as the unwanted and not enabled requests will not be processed. In other words, the remote communication process operates as a filter (or firewall) that filters out unwanted requests.

In some embodiments the at least one access criterion comprises that the sender or origin of the request is authenticated. Thereby, requests from unknown systems will be blocked.

In some embodiments the at least one access criterion comprises that the request has an allowed request type. Thereby, unknown or disabled access types will be blocked.

In some embodiments the at least one access criterion comprises that the request concerns an allowed functionality. Thereby, requests to access functionality for which remote access is disabled, will be blocked.

In some embodiments the at least one access criterion comprises that the number of received requests per time unit do not exceed a threshold value. Hence, if incoming requests are received at a very high rate, which means that they will potentially starve out the one or more medical processes, then they will not be forwarded to the one or more medical processes at this rate. By blocking requests received at a very high rate, the risk of starving out of the critical internal components may be mitigated.

In some embodiments the at least one access criterion comprises individual rules for different senders, types of requests and/or functionality. Thereby, it is possible to control which requests to allow in a precise way.

In some embodiments the configuring comprises receiving the configuration data from an operator and/or reading configuration data stored in the medical device and configuring the at least one access criterion based on the received configuration data. Hence, the operator may control how and by whom the medical device may be accessed. Alternatively, access may be controlled by a predefined configuration pre-loaded in the medical device, e.g. by manufacturer or owner.

In some embodiments the forwarding comprises forwarding the request using a protocol, which is different from and/or independent on the protocol used for the communication between the medical device and a remote system. Hence, the network used by the remote system is physically separated and non-routable to the internal network of the medical device.

In some embodiments the medical device comprises a set of processors and wherein the one or more medical processes and the remote communication process are both executed by the same processor or processors of the set of processors. Thus, the remote communication may be handled by the same processor that handles safety critical patient data, as long as the remote communication is executing in an isolated context. Advanced processors used in medical devices often have built-in support for communication with a remote system. Thus, as no additional processor is required to handle the remote communication, the proposed solution may be accomplished without addition of any extra hardware.

In some embodiments the request to access the one or more medical processes comprises a request to remotely control the medical device, a request to update software in the medical device, a request to set a time of the medical device, a request to receive log data from the medical device, a request to update a configuration of the medical device and/or a request to update credentials of the medical device. Hence, the proposed technique may be applied to different types of requests.

In some embodiments the method comprises giving the one or more medical processes higher priority to processing capacity of the medical device, than to the remote communication process. Thus, the operation of medical procedures and of the associated support procedures always have highest priority.

According to a second aspect, the disclosure relates to a medical device for performing a medical procedure, comprising, a set of processors, a set of memory units storing a software system for execution by the set of processors, and a communication interface enabling communication with one or more remote systems. The software system is configured to, when executed by the set of processors, operate the medical device to perform the medical procedure and associated support procedures. The software system comprises one or more medical processes involved in the operation of the medical device and a remote communication process, being separate from the one or more medical processes. The remote communication process is configured to manage communication between the medical device and one or more remote systems and the one or more medical processes have higher priority than the remote communication process. Furthermore, the software system, when executed by the set of processors, performs the method according to the first aspect.

According to a third aspect, the disclosure relates to a computer-readable medium comprising a software system for remotely accessing a medical device to perform a medical procedure. The software system comprises one or more medical processes involved in the operation of the medical device and a remote communication process that is separate from the one or more medical processes. The remote communication process is configured to manage communication between the medical device and one or more remote systems. Furthermore, the one or more medical processes have higher priority than the remote communication process. Furthermore, the software system, when executed by a set of processors of the medical device, performs the method according to the first aspect.

### BRIEF DESCRIPTION OF THE DRAWINGS

FIGS. 1a to 1c illustrate medical devices that may implement embodiments of the disclosed technique.

FIGS. 2a and 2b illustrate a control system of any of the medical devices of FIG. 1a to 1c in more detail.

FIG. 3 conceptually illustrates an example software system for remotely accessing a medical device.

FIG. 4 is a flow chart of a method for remotely accessing a medical device.

FIG. 5 is a sequence diagram of internal signalling between processes of the software system of a medical device when performing the proposed method according to one example implementation.

### DETAILED DESCRIPTION

Embodiments of the present invention will now be described more fully hereinafter with reference to the accompanying drawings, in which some, but not all, embodiments of the invention are shown. Indeed, the inventive solution may be embodied in many different forms and should not be construed as limited to the embodiments set forth herein; rather, these embodiments are provided so that this disclosure may satisfy applicable legal requirements. Like numbers refer to like elements throughout.

This disclosure proposes that all interactions and functionality in a medical device that manages communication with remote systems are architecturally isolated in a separated software process (or software component), herein called remote communication process, that executes in a separated context. This abstracts the details of cybersecurity away from internal software processes that handle the medical application and also makes the internal software processes agnostic to communication protocol specifics. The remote communication process component may also reduce the system load by only accepting the wanted and enabled requests. For example, in some embodiments requests from a remote system are only served and forwarded to internal software processes of the medical device at a rate that is safe for the medical device. Rejection of disabled requests and communication is for example done by defensively configuring a firewall of the medical device, which is an efficient design that reduces performance need. Hence, the proposed remote communication process differs from a standard interface in that it is isolated from the rest of the one or more medical processes and that it has lower priority. The remote communication process is not involved in executed in performing the medical procedure, but basically only handles the remote communication. All incoming requests must pass through the remote communication process. As a result, the medical procedure will not be affected, even if the remote communication process is attacked and harmed.

For better understanding of the proposed technique some example medical devices will first be described. A medical device is an automated apparatus or machine which is configured to be operated, optionally in combination with one or more other medical devices, to perform a medical procedure in relation to a human or animal subject. As used herein, a medical procedure may involve one or more of diagnosis, prevention, monitoring, treatment or alleviation of a disease, an injury, or a handicap, or monitoring for detection thereof.

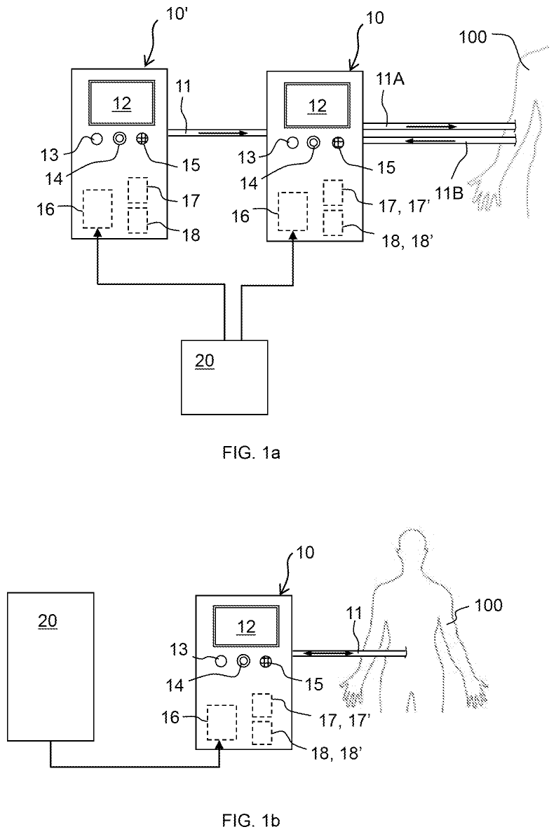

FIG. 1a illustrates two example medical devices 10, 10′ which may be involved in a medical procedure of performing extracorporeal blood treatment, e.g. as part of renal replacement therapy, such as hemodialysis, hemodiafiltration, hemofiltration or isolated ultrafiltration. The medical device denoted 10 is a blood treatment apparatus, which comprises a blood withdrawal line 11A and a blood return line 11B for connection to the circulatory system of a subject 100, e.g. at a blood vessel access. As indicated by arrows, the medical device 10 is operable to withdraw blood from the subject 100, process the blood in a dialyzer (not shown) and return the processed blood to the subject 100 in a controlled manner, by means of e.g. a blood pump. In the dialyzer, blood is passed on one side of a semipermeable membrane and dialysis fluid is passed on the other side of the membrane. The membrane allows waste particles and water to move from the blood to the dialysis fluid, and desired particles to move from the dialysis fluid to the blood. The blood treatment apparatus 10 may also include a syringe driven by a syringe pump, where the syringe is used for heparin infusion or calcium infusion. The medical device denoted 10′ is operable to prepare a fluid for use by the blood treatment apparatus 10 and comprises a fluid line 11 for supplying the fluid to the blood treatment apparatus 10. In one example, the medical device 10′ is a water preparation apparatus and the fluid is purified water. For example, the water treatment apparatus 10′ may filter incoming water by reverse osmosis as known in the art.

In the illustrated example, the medical device 10 comprises a display 12 (possibly a touch display), control buttons 13 (one shown), an indicator lamp 14, a loudspeaker 15, a control system 16, one or more actuators 17 for controlled withdrawal, processing and return of the blood to the subject 100 via the blood withdrawal line 11A and a blood return line 11B, and one or more sensors 18 for providing sensor data indicative of the medical procedure performed by the blood treatment apparatus. The medical procedure may also include for example priming and function checks. The actuator(s) 17 and sensor(s) 18 may include internal components (as indicated by dashed lines) or external components of the medical device 10, or both.

The actuators 17 are, for example, configured to control a valve, a pump and/or a heater while the medical procedure is performed. In other words, the actuators 17 are arranged to control the medical procedure. The actuator(s) 17 and sensor(s) 18 may also comprise auxiliary sensors 18′ and auxiliary actuators 17′ used to supervise the medical procedure and possibly take preventive action. The auxiliary actuators 17′ are for example configured to control an emergency valve or emergency brake to interrupt the medical procedure. To change e.g. a fluid flow rate the user typically inputs a set value for the fluid flow via for example a Graphical User Interface, GUI, generated on the display 12 or using the control buttons 13. This set value is then translated to one or more actuator set values, i.e. the values that controls the corresponding actuators. For example, a fluid flow rate of 300 ml/min (set value) is translated in pump rate in e.g. rpm or percent (actuator set value).

The control system 16 is configured to coordinate the operation of the actuator(s) 17 and the sensor(s) 18 to perform the intended medical procedure of the blood treatment apparatus, as well as to operate the display 12, the indicator lamp 14 and the loudspeaker 15 as needed in connection with the medical procedure, and to obtain user input via the control buttons 13. For example, the display 12 may be operated to present instructions to the user of the medical device 10, the indicator lamp 14 may be operated to indicate a medical device status, and loudspeaker 15 may be operated to generate an alarm signal, etc.

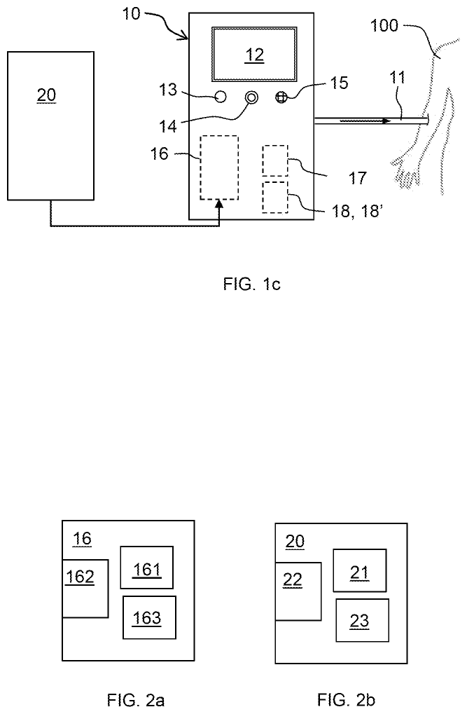

The medical device 10 is connected to a remote system 20 (only one illustrated but it may be a plurality). The remote system 20 is e.g. configured to remotely monitor and/or control the medical device. The remote system 20 will be further described in FIG. 2b.

The medical device 10′ may have a similar set of components as the medical device 10, on the illustrated level of detail, and will not be described further. The medical device 10′ is also connected to a remote system 20, in the same manner as the medical device 10. The medical devices 10, 10′ may comprise more components than illustrated that will not be explained here for brevity.

FIG. 1b illustrates another example medical device 10 which is operable to, in a controlled manner, deliver a dialysis fluid to the abdominal cavity of a subject 100 and subsequently remove the dialysis fluid therefrom, as indicated by a double-ended arrow. This medical procedure is commonly known as automated peritoneal dialysis, and the medical device 10 is often denoted a “PD cycler”. The PD cycler comprises, for example, a pump for any of mixing, delivering and removing dialysis fluid. The medical device 10 in FIG. 1b may have a similar (but typically reduced) set of components compared to the medical device 10 in FIG. 1a, on the illustrated level of detail and may also be connected to a remote system 20. The medical device 10 in FIG. 1b may comprise more or less components than illustrated that will not be explained here for brevity. The medical device 10 may also be connected to another medical device 10′ as illustrated in FIG. 1a, for obtaining purified water used for mixing dialysis fluid.

FIG. 1c illustrates a further example medical device 10, which is operable to deliver a medical fluid into the body of a subject 100, such as a human, in a controlled manner, as indicated by an arrow, e.g. into the circulatory system of the subject 100. The medical fluid may be any suitable liquid, including but not limited to medication and/or nutrients. This type of medical device 10 is commonly known as an “infusion pump”. One or more actuators of the infusion pump is configured to control one or more pumps to pump medication and/or nutrients at a specified rate into the subject 100. Line occluders and/or valves are actuated for controlling the fluid flow during the pumping. The medical device 10 in FIG. 1c may have a similar (but typically reduced) set of components and may be connected to a remote system 20 as the medical device 10 in FIG. 1b, on the illustrated level of detail, and will not be described further. The medical device 10 in FIG. 1c may comprise more components than illustrated that will not be explained here for brevity.

FIG. 2a illustrates the control system 16 of any of the example medical devices of FIG. 1a-c in more detail according to one example embodiment. The control system 16 comprises a processor 161, a communication interface 162 and memory 163. The processor 161 may be any commercially available processing device, such as a Central processing unit (CPU), Digital Signal Processor (DSP), a microprocessor, microcontroller, a Field-programmable gate array (FPGA), an Application-specific integrated circuit (ASIC), or a combination thereof.

The communication interface 162 is configured to enable communication with the remote system 20 (FIG. 1a-c). The communication may be wireless and/or wired. Wired communication may be performed using a wired Ethernet connection, RS-232, RS-485 or UART. Wireless communication may be performed via any of Bluetooth™, WiFi™, Zigbee®, Z-Wave®, wireless Universal Serial Bus (“USB”), or infrared protocols, or via any other suitable wireless communication technology. The communication interface 162 is for example a Bluetooth™ chip, configured to be controlled by the processor 161. The communication between the remote system 20 and the medical device may be performed using any suitable communication protocol, such as Internet Protocol or a proprietary protocol. In some embodiments, the communication interface may implement secure communication e.g. using pre-shared key approach, secure DNS communication using SSL/TLS, secure DHCP communication using delayed authentication and pre-shared key and or other application specific protocols using SSL/TLS.

The memory 163 may include non-volatile memory or volatile memory, or a combination thereof, including but not limited Read-Only Memory (ROM), Programmable Read-Only Memory (PROM), Electrically Erasable Programmable Read-Only Memory (EEPROM), flash memory, removable memory, Random-access memory (RAM), Dynamic random-access memory (DRAM), Static RAM, cache memory, hard drive, storage medium, etc. The memory 163 stores a software system for execution by the processor 161. The software system is an integrated collection of software items organized to accomplish a specific function or set of functions, see ISO standard IEC 62304 regarding medical devices. The software system comprises application software and a software platform (typically including an operating system) that manages the software applications and acts as an intermediary between the applications and the hardware of the medical device. The software system may be accessed by remote systems 20 via the communication interface 162.

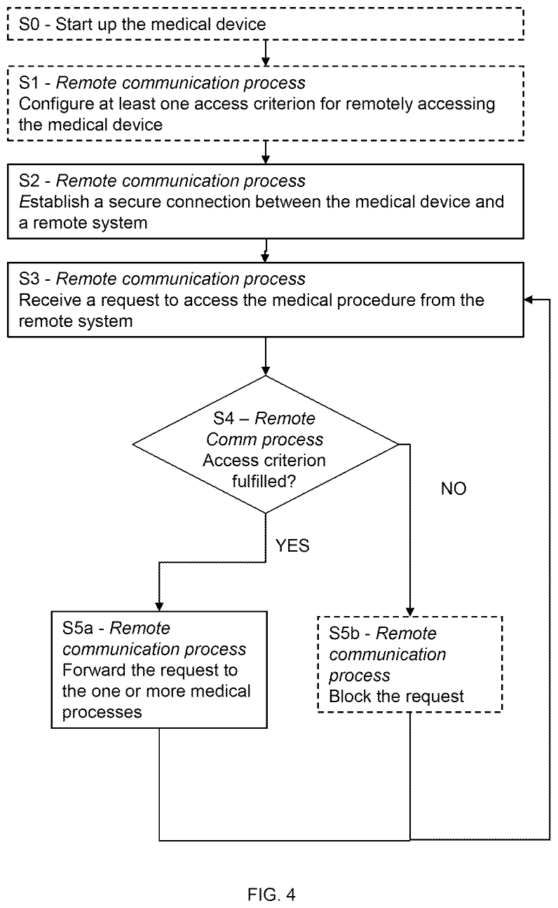

The software system is specified by one or more instructions stored in the memory 163 that are executable by the processor 161 to perform the operations described herein. The software system is configured to control the medical device 10 to perform e.g. the medical procedure. In other words, the software system includes one or more medical processes P_{MED}, involved in the operation of the medical device 10. The software system is also configured to, when executed by the processor 161, perform the method of the first aspect (FIG. 4), which will be described in detail below.

The processor 161 and memory 163 are “separate” in the sense that they are individually operable units, while they may or may not be located in any combination on a common substrate, e.g. in an integrated circuit. For simplicity, the control system 16 of FIG. 2a is illustrated as comprising only one processor 161, and memory 163. However, it should be appreciated that the medical device 10 may comprise a set of processors comprising one or more processors 161 and that the memory 163 may be implemented by one or several memory devices.

FIG. 2b illustrates an example remote system 20 in further detail. A remote system 20 is any system that is not an integrated part of the medical device, or more specifically of the software system executed by the set of processors 161 of the control system 16 of the medical device 10. The remote system 20 is e.g. a service or support system. The remote system 20 may comprise one or more of a server, workstation laptop, computer etc. The remote system may be located on-site or off-site. In its simplest form, the remote system is a simple user device such as a tablet or personal computer, which has software configured to remotely access the medical device 10 installed thereon. The remote system 20 comprises a processor 21, a communication interface 22 and memory 23. The processor 21 may be any commercially available processing device, such as a CPU, DSP, a microprocessor, an FPGA, an ASIC, or any other electronic programmable logic device, or a combination thereof.

The communication interface 22 is configured to enable communication with the medical device 10. The communication may be wireless and/or wired. Wired communication may be performed using a wired Ethernet connection, RS-232, RS-485 or UART. Wireless communication may be performed via any of Bluetooth™, WiFi™, Zigbee®, Z-Wave®, wireless Universal Serial Bus (“USB”), or infrared protocols, or via any other suitable wireless communication technology. The short-range communication interface 22 is for example a Bluetooth™ chip, configured to be controlled by the processor 21. The communication between the remote system 20 and the medical device 10 may be performed using any suitable communication protocol, such as Internet Protocol or a proprietary protocol.

The memory 23 may include non-volatile memory or volatile memory, or a combination thereof, including but not limited to ROM, PROM, EEPROM, flash memory, removable memory, RAM, DRAM, SRAM, cache memory, hard drive, storage medium, etc. The memory 23 store software for execution by the processor 21. The software is e.g. configured to control the medical device 10 to perform e.g. service, support.

For simplicity, the remote system 20 of FIG. 2b is illustrated as comprising only one processor 21, and memory 23. However, it should be appreciated that the remote system 20 may comprise several processors and that the memory 23 may be implemented by one or several memory devices.

The remote system 20 is configured to receive information e.g. sensor data provided by the sensors 18, from the medical device 10, via the communication interface 22. The remote system 20 may also send information to the medical device 10 e.g. to remotely access the medical device 10 for different purposes. The communication is typically implemented using messages communicated via the communication interface 22. The messages may be indicative of different commands (access requests) or they may carry data (e.g. remote requests).

In order to separate treatment from other types of operation, medical devices can often be operated in different modes, such as therapy mode and service mode. The service mode is typically used for procedures that are not treatment, e.g. service or support. One example of remote access is that the remote system 20 may access the medical device 10 to cause the medical device 10 to be set in service mode. Another example, is that the remote system 20 may request the medical device 10 to send service mode data to the remote system 20 or change a setting such as language, alarm-threshold etc. The service mode data may include the present settings, information of the latest performed maintenance, information of the installed software version etc. of the medical device 10.

Remote access may also be used by a remote system 20 to perform a software update on the medical device 10. This may be done by sending information that updated software is available, which may also be done in a therapy mode. An operator may then be informed about the available software update e.g. via the display 12. The operator may then trigger downloading and installation of the software update at a suitable time. Remote access may also be used to install the updated software on the medical device 10.

Another example of remote access is remote access of actuators 17 or internal procedures of the medical device for different purposes. In practice it is possible to perform any procedure of the medical device 10 remotely, even the medical procedure itself.

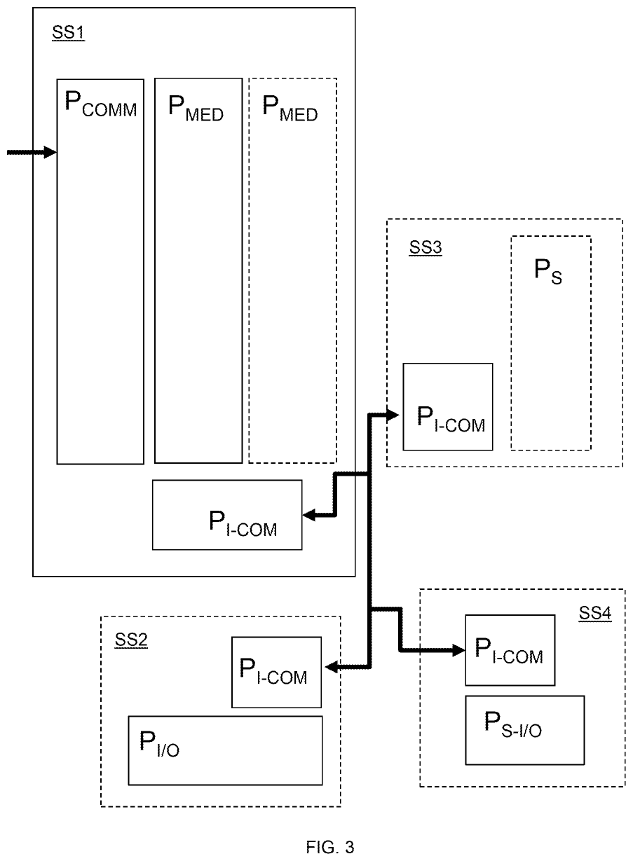

The proposed technique will now be described with reference to FIG. 3 to FIG. 5.

FIG. 3 is a block diagrams of an example software system configured to control the medical device 10, in accordance with a non-limiting example of the proposed technique.

The illustrated software system comprises four subsystems, denoted SS1-SS4. The subsystems SS1-SS4 may be considered to be software modules of computer-executable instructions that may be independently developed and tested to provide specific functionality in relation to the operation of medical device when performing the medical procedure and the support procedures. Each respective subsystem comprises software processes (or threads) executed within the context of the respective subsystem. The software processes within a subsystem may thus be designed to perform a group of coordinated processes to provide the specific functionality of the subsystem. A software process (or simply process) herein refers to a software component comprising a sequence of instructions that can be managed independently by a scheduler, which is typically a part of an operating system of the software system executed by the processor(s) 161 of the control system 16 of the medical device 10. The implementation of processes differs between operating systems. Different processes do typically not share resources such as memory spaces. The software processes are e.g. Linux processes or Green Hills Integrity processes. The operating system typically comprises a standard firewall, such as Netfilter, that monitors and controls incoming and outgoing network traffic based on predetermined security rules.

It is also conceivable that a subsystem includes one or more processes that are not involved in performing the medical procedure. Further, a subsystem may include further software components, such as middleware and/or low-level software components that perform basic functions or services in the software system, such as a communication stack for managing communication with other subsystems, an error manager for managing technical errors, a notification manager for managing notifications, etc. One or more of the subsystems may be operated on top of one or more operating systems to make use of services provided by the operating system(s) in relation to hardware and software resources. Depending on implementation, the operating system(s) of the sub systems of the software system executed by the processor(s) 161 may, e.g., include a real-time operating system, an embedded operating system, a single-tasking operating system, or a multi-tasking operating system, or any combination thereof.

Software processes associated with the proposed technique will be described with reference to the example architecture of FIG. 3. The first subsystem SS1 comprises one or more medical processes, P_{MED}, configured to control the operation of the medical device e.g. to perform a medical procedure, such as a dialysis treatment, or a support procedure. Thus, the one or more medical processes, P_{MED}, may also involve processes configured to control operations of the medical device 10 associated with the medical procedure such as support and/or service. Therefore, first subsystem SS1 is herein referred to as the main system of the medical device 10.

The main system SS1 also comprises a remote communication process P_{COMM}. The remote communication process P_{COMM} is configured to manage communication between the medical device 10 and one or more remote systems 20. In other words, the remote communication process P_{COMM} is responsible for establishing a secure (authenticated and encrypted, where applicable) connection to the remote system 20. The remote communication process P_{COMM} is also responsible for communicating with the remote system 20. As all communication with the remote system 20 is handled by the remote communication process P_{COMM} in the main system SS1, remote access to functionality of the one or more medical processes P_{MED} always goes via the remote communication process P_{COMM}. In other words, all requests to remotely access the one or more medical processes P_{MED} or other processes of the main system are routed through (i.e. are received via or enters through) the remote communication process P_{COMM}.

It should be appreciated that even if the medical device 10 used to illustrate the proposed technique comprises one single remote communication process P_{COMM}, in other examples there may be two or more remote communication processes P_{COMM}. For example, the medical device may comprise a plurality of remote communication processes P_{COMM} for handling different types of remote requests and/or protocols. In this case each one of these remote communication processes P_{COMM} must be separated from the one or more medical processes P_{MED} and has lower priority than the one or more medical processes P_{MED}, as explained above.

The remote communication process P_{COMM} is separate from the one or more medical processes P_{MED}. That the processes are separated means that they have, for example, separate memory spaces, individual process states and/or are communicating using inter process communication. Stated differently, the idea is to isolate one process (or possibly a plurality of processes), here P_{COMM}, that handles remote communication, which is limited in terms of what it is allowed to do. In some embodiments, remote communication process P_{COMM} does not have access to system resources except those needed for communication with the remote system. In some embodiments, its only task is to serve the enabled requests. From now on only one remote communication process P_{COMM} will be discussed. However, if there are several remote communication processes P_{COMM}, the same requirements apply to each one of them.

As mentioned above in connection to FIG. 2a, the medical device 10 may comprise one or several processors 161. As the processes are separated, the one or more medical processes P_{MED} and the remote communication process P_{COMM} may be executed by the same processor or processors, and still remain isolated.

In other words, an error in one of the processes will not propagate to the other process.

Furthermore, the one or more medical processes P_{MED} have higher priority than the remote communication process P_{COMM}. This means that the software system always prioritises the one or more medical processes P_{MED} in terms of e.g. processing power and memory access. More specifically, each process is assigned a priority. The concept of process priority is well known within computer science. The concept means that the process with highest priority is to be executed, or assigned resources, before any process with lower priority. Processes with same priority are for example executed on first come first served basis. Priority can be decided based on memory requirements, time requirements or any other resource requirement. Hence, if there is an overload in the remote communication process P_{COMM}, then it will not affect the one or more medical processes P_{MED}.

As a further example, the remote communication process P_{COMM} is not allowed to access certain files. However, the one or more medical processes P_{MED} may be permitted to access the certain files.

Many medical devices that perform a medical procedure that pose a health risk to a subject in case of operational failure in the main system that controls the medical procedure, or any of the hardware components used by such a main system, may include a protective system (also referred to as a supervision system) that operates in parallel and is independent of the main system. Whenever the protective system detects a potential operational failure, the medical device is switched to a safe operating state. Thus, in this example the third subsystem SS3 comprises a supervision process P_{S} configured to supervise the operation of the medical procedure. The third subsystem SS3 is herein also referred to as the protective system of the medical device 10.

The subsystems SS2 and SS4 are I/O systems configured to communicate with different sets of actuators 17 and/or sensors 18 of the medical device 10 based on commands/signals generated by the main system SS1 and the protective system SS3, respectively. To this end, each of the subsystems SS2, SS4 may comprise a software process for providing access to peripherals (e.g. actuators 17 and/or sensors 18 (FIG. 1a-c)) connected to the respective subsystem SS2, SS4. In FIG. 3 these processes are denoted P_{I/O} (short for I/O process) and P_{S-I/O} (short for supervision I/O process).

The processes of the sub systems SS1-SS4 communicate using inter-process communication, IPC, which refers to mechanisms of an operating system that allow the processes to manage shared data. The inter process communication typically uses a message-based protocol. For example, inter-process communication use clients and servers, where the client requests data and servers respond to client's requests. The design of the inter-process communication is up to implementation.

In this example, each subsystem also comprises a respective internal communication process P_{I-COM}. The communication process P_{I-COM} are responsible for the routing requests to correct receivers and maintaining the active communication session, e.g. it handles communication between processes of the different sub systems SS1 to SS4. As all communication with the remote system 20 is performed via the remote communication process P_{COMM} in the main system SS1, remote access to functionality of the other subsystems SS2-SS4 is handled by the communication processes P_{I-COM}. In other words, all requests to remotely access the medical device 10 are routed or passed through the remote communication process P_{COMM} of the main system SS1.

Note that the proposed technique may as well be implemented in medical devices that do not comprise a protective system SS3 and corresponding I/O system, SS4. Also, it is possible to have a design where the I/O systems are not separate sub systems but are integrated in the main system SS1 and the protective system SS3 (if present). Therefore, the subsystems SS2, SS3, SS4 are illustrated with dashed lines in FIG. 3.

The proposed technique will now be described in further detail with reference to the flowcharts of FIG. 4. FIG. 4 illustrates an example method for remotely accessing a medical device 10 configured to perform a medical procedure, such as the medical device 10 of any of the FIGS. 1a-1c. The method is e.g. performed in a medical device 10 located in a medical care environment, such as a hospital, where the medical device 10 is used to e.g. treat patients. The method may also be performed in a medical device 10 at a patient's home. The method makes it possible to enable a remote system, located inside or outside the medical care environment (and an operator of such a system), to access the machine e.g. to diagnose the medical device 10 in a secure way.

As described above, the medical device 10 is controlled by a software system, one or more medical processes P_{MED} involved in the operation of the medical device 10, and a remote communication process P_{COMM}. The remote communication process P_{COMM} is configured to manage communication between the medical device 10 and one or more remote systems 20. The remote communication process P_{COMM} is separate from the one or more medical processes P_{MED}. Furthermore, the one or more medical processes P_{MED} have higher priority than the remote communication process P_{COMM}, as explained in connection with FIG. 3.

The method is typically implemented as a computer program comprising instructions which, when the program is executed by a set of processors, cause the computer to carry out the method. According to some embodiments, the computer program is stored in a computer-readable medium that comprises instructions which, when executed by a set of processors, cause the computer to carry out the method.

The method may be performed anytime when the medical device 10 is switched on. A prerequisite for being able to perform the method is typically that that the communication interface 162 of the medical device 10 is enabled.

When the medical device is switched on a start-up procedure is initiated. In some embodiments the method comprises the step of starting up S0 the medical device 10, which is typically done when the medical device 10 is powered on. All requests (i.e. incoming request messages) from the remote system 20 are typically denied until the start-up procedure is completed. In other words, in some embodiments, requests to access the one or more medical processes P_{MED} are blocked by the remote communication process P_{COMM} during the starting up S0. That the requests are blocked means that they are disregarded, or possibly stored, until the start-up procedure is complete.

During start-up, the remote communication process P_{COMM} configures S1 at least one access criterion for remotely accessing the medical device 10. For example, communication settings of the remote system 20 are configured in the medical device 10. The configuration data may be received from an operator via a GUI of the medical device 10. Alternatively, configuration data stored in the medical device 10 may be read by the remote communication process P_{COMM}. Thus, configuration data may be added at manufacturing or when the medical device 10 is put into use.

The communication settings e.g. comprise IP addresses of remote systems that the medical device 10 needs to access. It may also comprise addresses of remote systems 20 that are allowed to access the medical device. In this example, the IP address of the remote system 20 may be configured in the medical device 10. In some embodiments, devices that are not registered would not be permitted to access the medical device 10.

Additionally or alternatively, the communication settings may comprise authentication data to verify the authenticity of the remote system 20. In other words, in some embodiments, credentials that the remote system 20 uses are configured in the medical device 10. It may include e.g. credentials configured in the medical device 10 and shared by the remote system 20, e.g. using private-public-key cryptography. In this way the medical device 10 may assure that a particular remote system 20 is trusted and should be allowed to access the medical device 10. In other words, in some embodiments, the at least one access criterion comprises that the sender or origin of the request is authenticated. For example, the request to remotely access the medical device 10 has to be signed with a cryptographic key known to the medical device 10 in order to be accepted.

Furthermore, certain functionality of the medical device is enabled for remote access by a remote system 20. For example, the remote system 20 is allowed to read certain sensors 18, control certain actuators 17, or execute certain commands. In other words, the at least one access criterion comprises that the request has an allowed request type or that the request concerns an allowed functionality. For example, allowed request types may include a request to make a SW update or a request comprises a request to receive log data from the medical device 10.

In other words, in some embodiments the at least one access criterion comprises individual rules for different senders, types of requests and/or functionality.

The remote communication process P_{COMM} may also be configured not to forward requests at a rate that may harm the system. Stated differently, the remote communication process P_{COMM} may be configured to always forward requests at a safe rate. In this way, it is avoided that the other processes of the medical device 10 are starved in case of an overloading attack. Resource starvation is a problem encountered in concurrent computing, where for example an operating system constantly fails to provide a process necessary resources, such as memory or computing resources) that a process requires to perform its tasks. Starvation of a medical device 10 can be intentionally caused by a remote system 20 by sending a large number of requests to the medical device 10, making the medical device 10 allocate its resources to handling of the requests instead of performing the medical procedure. In other words, in some embodiments the at least one access criterion comprises that the number of received requests per time unit do not exceed a threshold value. The threshold is for example a number of requests per second depending on a reception capacity of the remote communication process P_{COMM}. Typically, about one or more dozen per second. The rate may also be defined per request type or functionality.

The configuring S1 may be done when setting up the system, or it may be done at a later point in time. In some embodiments, the medical device 10 is re-configured e.g. another remote system may be configured by an operator via the GUI to be an allowed (or trusted) remote system when required.

In order to remotely access the medical device a connection needs to be established between the medical device 10 and the remote system 20. The connection is typically a secure connection established for exchange by exchange of credentials or by using a VPN connection. The connection may also be encrypted. More specifically, a remote connection is established between the communication interface 162 of the medical device 10 and the communication interface of the remote system 22. The connection is e.g. an IP connection that may use different underlying transport protocol. The connection may use wireless or wired communication or a combination thereof. In some embodiments the connection is established over the internet with a remote system 20 located e.g. in another location. Hence, the connection may be subject to cyberattacks. In other words, the method comprises that the remote communication process P_{COMM} establishes S2 a secure connection between the medical device 10 and a remote system 20.

When the remote system 20 wants to access the medical device 10, a request is typically sent to the medical device 10. The request is received by the remote communication process P_{COMM} Thus, the method comprises the remote communication process P_{COMM} receiving S3 from the remote system, a request (that is a message) to access the one or more medical processes P_{MED}. The medical device may support requests having different formats, depending on the purpose. Typically, the medical device 10 has one or more protocols that the remote system 20 is configured to support. However, the request typically comprises a request type and possibly also (or alternatively) an internal destination within the medical device, such as a sensor or an internal function. In addition, the request may comprise data such as set values or other parameters.

Different types of requests may be received from the remote system 20. In some embodiments the request to access the one or more medical processes comprises a request to remotely control the medical device 10. Remote control may e.g. be used for service or support of the medical device 10. In some embodiments the request to the one or more medical processes P_{MED} comprises a request to update software in the medical device 10. This type of access may require extra security measures. In some embodiments the request to access the one or more medical processes comprises a request to set a time of the medical device 10 or to receive log data from the medical device 10. In some embodiments the request to access the one or more medical processes comprises a request to update a configuration of the medical device 10. The configurations may be related to remote access configurations (see step S1). In some embodiments the request to access the one or more medical processes comprises a request to update credentials of the medical device 10.

However, the remote communication process P_{COMM} will not forward any requests that do not fulfill the configured at least one access criterion. Hence, the method comprises that the remote communication process P_{COMM} evaluates S4 whether the received request fulfils the configured at least one access criterion for remotely accessing the medical device 10.

If the request fulfills the predetermined criteria, e.g. if the remote system 20 can authenticate itself and the request is received at a safe rate, the request will be forwarded to relevant software processes. In other words, the method comprises that the remote communication process P_{COMM} forwards S5a the request to the one or more medical processes P_{MED}, upon the request fulfilling the configured at least one access criterion for remotely accessing the medical device 10. Stated differently, the remote communication process P_{COMM} includes a filter for incoming requests.

The remote communication process P_{COMM} typically receives request from the remote system using a standardized protocol e.g. IP. However, another protocol e.g. a proprietary protocol of a manufacturer, may typically be used internally within the machine. In other words, in some embodiments the remote communication process P_{COMM} forwards the request using a protocol, which is different from and/or independent on the protocol used for the communication between the medical device 10 and a remote system 20. This means that external communication with the remote system 20 and the internal communication within the medical device 10 are not directly routable, but always goes through the remote communication process P_{COMM}, which translates and/or converts the requests to a protocol used for internal communication and also checks that the at least one configured access criterion is fulfilled.

As explained above, it is crucial that the one or more medical processes P_{MED} are not starved in situations when the remote communication process P_{COMM} requires a significant amount of processing capacity. Hence, the operation of the one or more medical processes P_{MED} is of the highest priority. In other words, in some embodiments the method comprises giving the one or more medical processes P_{MED} higher priority to processing capacity of the medical device 10, than to the remote communication process P_{COMM}. This may be implemented by assigning a higher priority to the one or more medical processes P_{MED} in the operating system.

Requests that do not fulfill the access criteria are typically blocked by the process P_{COMM}. In other words, in some embodiments the method comprises that the remote communication process P_{COMM} blocks S5b requests to access the one or more medical processes P_{MED} that fail to fulfil the configured at least one access criterion. Blocking implies that they are not forwarded to other processes within the medical device 10. In some scenarios the blocking may be temporary. For example, if requests are received at an unsafe rate, then the requests may be blocked for a while (e.g. in a buffer), and then forwarded at a safe rate. Thus, the time in the buffer depends on the incoming rate and the safe rate.

The remote system 20 is typically not informed if the requests are blocked, as it is undesirable that a malicious remote system gets that type of information.

Steps S3 to S5 may then be repeated for incoming requests to access the medical device 10, until the secure connection between the medical device 10 and the remote system 20 is terminated, e.g. via the GUI or because the connection is otherwise broken. The connection may also (or alternatively) be automatically ended/terminated when the “transaction” between the remote system 20 and the medical device 10 is completed.

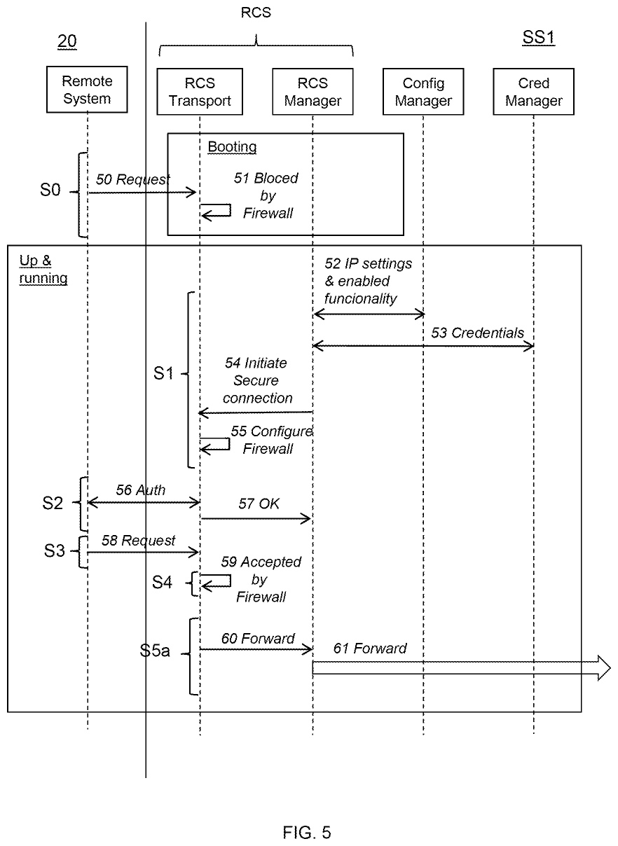

FIG. 5 is a sequence diagram of internal signalling between processes of the main system SS1 of a medical device 10 when performing the proposed method according to an example implementation.

This example implementation also refers to the medical device of FIG. 1a-1c and the example software architectures (in particular main system SS1) described in connection with FIG. 3 above.

The first subsystem, also referred to as main system, SS1 is implemented by a plurality of SW items that manage different functionality of the medical device 10. A SW item is a functional part of the software architecture. A SW item can be deployed by one or several software processes. Alternatively, a software process may implement one or several SW items. For simplicity, only SW items that are involved in the proposed method for remotely accessing the medical device are illustrated in FIG. 5.

The main system SS1 comprises, in addition to the one or more medical processes P_{MED} (not shown in FIG. 5), Remote System Communication, a Configuration Manager, and a Credential Manager. In this example, all processes, except RSC, which is implemented by two SW items, are implemented by one corresponding SW item.

The Configuration Manager is responsible for managing the configuration parameters in the system. The Configuration Manager stores the configuration parameters, updates them on request, logs updates, and provides the configuration parameters to clients.

The Credentials Manager is responsible for storing cybersecurity credentials such as certificates, private and public keys as well as providing the credentials to clients.

Remote System Communication, RSC, corresponds to the remote communication process P_{COMM}, described above. Thus, RSC is responsible for establishing a secure (authenticated and encrypted, where applicable) connection to the remote system 20. RSC is also responsible for communicating data with the remote system 20. In this example implementation, RSC is divided into the following SW items:
RSC Manager: Responsible for activating the enabled functionality (retrieved from Configuration Manager) towards remote systems and converting it to internal protocols.
RSC Transport: Responsible for the external protocol formatting and encryption, in general.

Remote System Communication is typically a client to device internal SW items. Hence, the Remote System Communication does not act a server, since the medical device internal SW items are typically not dependent on the existence of Remote System Communication.

An example of signalling between processes in the main system SS1 during secure connection setup will now be described with reference to FIG. 5.

Some prerequisites typically need to be fulfilled before the activation is initiated. For example, IP (Internet Protocol) settings should have been configured in the Configuration Manager e.g. via GUI. In addition, certain functionality should typically be enabled/disabled for remote access either via the remote system 20 or GUI. Alternatively, there may be default configurations that may be used. In addition, the cryptographic keys to be used by the secure connection are installed on the medical device, e.g. at manufacturing.

Initially, while booting the medical device 10, all requests received (step 50) from the remote system 20 are typically blocked (step 51) by the firewall (in RSC Transport). These steps correspond to step S0 of FIG. 4.

Once the medical device 10 is up and running (i.e. the booting is complete), the RSC Manager retrieves (step 52) the IP settings and enabled communication functionality from Configuration Manager and retrieves (step 53) the credentials from Credentials Manager. The RSC Manager then requests (step 54) RSC Transport to initiate a secure connection, with credentials and enabled communication functionality provided. Thus, RSC Transport configures (step 55) the firewall to only accept the ports and protocols used by currently enabled communication functionality, received from the RSC Manager. The at least one access criterion for remotely accessing the medical device 10 are now configured in RSC Transport. Hence, these steps correspond to step S1 of FIG. 4.

To establish the secure connection, the remote system 20 first needs to authenticate itself using the credentials received from RSC Manager (step 56). Thereby, a secure connection is established with the remote system 20, and the RSC Manager is informed (step 57). Thus, these steps correspond to step S2 of FIG. 4.

The medical device now accepts access requests that fulfil the configured at least one access criterion. Thus, when a request is received (step 58), it will be accepted by the firewall (step 59) and forwarded to RSC Manager (step 60) provided that it matches the configured IP settings and enabled functionality. These steps correspond to steps S3 and S4 of FIG. 4.

RSC Manager then forwards (step 61), or passes on, the request to the correct recipient (typically another process) within the medical device 10. For example, the request is forwarded to the one or more medical processes P_{MED} or to one of the other sub systems SS2-SS4. This step corresponds to step S5a of FIG. 4.

While the present invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and the scope of the appended claims.

1-13. (canceled)

14: A method for remotely accessing a medical device configured to perform a medical procedure, wherein the medical device is controlled by a software system comprising one or more medical processes involved in the operation of the medical device and a remote communication process, the remote communication process separate from the one or more medical processes, wherein the remote communication process is configured to manage communication between the medical device and one or more remote systems, and wherein the one or more medical processes have a higher priority than the remote communication process, the method comprising:
configuring at least one access criterion for remotely accessing the medical device;
establishing a secure connection between the medical device and a remote system;
receiving, from the remote system, a request to access the one or more medical processes; and
forwarding the request to the one or more medical processes, in response to the request fulfilling the configured at least one access criterion for remotely accessing the medical device.

15: The method according to claim 14, wherein the method comprises starting up the medical device, wherein all requests to access the one or more medical processes are blocked by the remote communication process during the start up.

16: The method according to claim 14, wherein the method comprises blocking requests to access the one or more medical processes that fail to fulfill the configured at least one access criterion.

17: The method according to claim 14, wherein the at least one access criterion comprises at least one of: (i) an authentication of a sender or origin of the request, (ii) that the request includes an allowed request type, (iii) that the request relates to an allowed functionality, and (iv) that a number of received requests per time unit does not exceed a threshold value.

18: The method according to claim 14, wherein the at least one access criterion comprises individual rules for different senders, types of requests and/or functionality.

19: The method according to claim 14, wherein configuring the at least one access criterion comprises receiving configuration data from an operator and/or reading configuration data stored in the medical device and configuring the at least one access criterion based on the configuration data.

20: The method according to claim 14, wherein forwarding the request comprises forwarding the request using a protocol that is different from and/or independent of a protocol used for the communication between the medical device and the remote system.

21: The method according to claim 14, wherein the medical device comprises at least one memory in communication with one or more processor, the one or more processor configured to perform the one or more medical processes and the remote communication process of the software system.

22: The method according to claim 14, wherein the medical device comprises a set of processors, and wherein the one or more medical processes and the remote communication process are executed by the same processor or processors of the set of processors.

23: The method according to claim 14, wherein the remote communication process and the one or more medical processes are separate such that they have separate memory spaces, individual process states and/or communicate using inter process communication.

24: The method according to claim 14, wherein the request to access the one or more medical processes comprises at least one of: a request to remotely control the medical device, a request to update software in the medical device, a request to set a time of the medical device, a request to receive log data from the medical device, a request to update a configuration of the medical device, and a request to update credentials of the medical device.

25: The method according to claim 14, wherein having a higher priority includes giving the one or more medical processes a higher priority to a processing capacity of the medical device than the remote communication process.

26: A medical device for performing a medical procedure, comprising:
a set of memory units storing a software system configured to operate the medical device;
a set of processors in communication with the set of memory units, the set of processors configured to execute the software system; and
a communication interface configured to enable communication between the medical device and one or more remote systems,
wherein the software system comprises one or more medical processes involved in the operation of the medical device, and a remote communication process that is separate from the one or more medical processes, wherein the remote communication process is configured to manage communication between the medical device and one or more remote systems, and wherein the the one or more medical processes has a higher priority than the remote communication process.

27: The medical device of claim 26, wherein the one or more medical processes and the remote communication process are executed by the same processor or processors of the set of processors.

28: The medical device of claim 26, wherein the request to access the one or more medical processes comprises at least one of: a request to remotely control the medical device, a request to update software in the medical device, a request to set a time of the medical device, a request to receive log data from the medical device, a request to update a configuration of the medical device, and a request to update credentials of the medical device.

29: The medical device of claim 26, wherein the remote communication process and the one or more medical processes are separate such that they have separate memory spaces, individual process states and/or communicate using inter process communication.

30: The medical device of claim 26, wherein having a higher priority includes the one or more medical processes having a higher priority to a processing capacity of the set of processors than the remote communication process.

31: A non-transitory, computer-readable medium storing a software system for remotely accessing a medical device, which when executed by a processor, causes the processor to:
configure at least one access criterion for remotely accessing the medical device;
establish a secure connection between the medical device and a remote system;
receive, from the remote system, a request to access one or more medical processes of the system; and
forward the request to the one or more medical processes, in response to the request fulfilling the configured at least one access criterion for remotely accessing the medical device,
wherein the software system comprises:
the one or more medical processes, which are involved in the operation of the medical device, and
a remote communication process that is separate from the one or more medical processes, wherein the remote communication process is configured to manage communication between the medical device and one or more remote systems,
wherein the one or more medical processes have a higher priority than the remote communication process.

32: The non-transitory, computer-readable medium of claim 31, wherein the at least one access criterion comprises at least one of: (i) an authentication of a sender or origin of the request, (ii) that the request includes an allowed request type, (iii) that the request relates to an allowed functionality, and (iv) that a number of received requests per time unit does not exceed a threshold value.

33: The non-transitory, computer-readable medium of claim 31, wherein forwarding the request comprises forwarding the request using a protocol that is different from and/or independent of a protocol used for the communication between the medical device and the one or more remote systems.
